(19) United States
(12) Patent Application Publication
(10) Pub. No.: US 2026/0043777 A1
(43) Pub. Date: Feb. 12, 2026

(54) DETERMINATION METHOD, DETERMINATION DEVICE, AND RECORDING MEDIUM

(71) Applicant: SUMITOMO CHEMICAL COMPANY, LIMITED, Tokyo (JP)

(72) Inventors: Yuki KODAMA, Osaka-shi (JP); Michiaki ARITA, Chuo-ku (JP)

(73) Assignee: SUMITOMO CHEMICAL COMPANY, LIMITED, Tokyo (JP)

(21) Appl. No.: 19/290,637

(22) Filed: Aug. 5, 2025

(30) Foreign Application Priority Data
Aug. 9, 2024 (JP) 2024-134679

(51) Int. Cl. G01N 33/00; A61B 10/00

(52) U.S. Cl. CPC G01N 33/0001; A61B 10/007

(57) ABSTRACT

Provided are a determination method and the like, in which the health condition of a target can be determined by using an olfactory sensor.

The determination method causing a computer to execute processing of: acquiring a time-dependent response signal with respect to a target subject derived from a determination target, detected by using an olfactory sensor including cells that have olfactory receptors and respond to odorous molecules; and determining a health condition of the determination target, on the basis of a profile of the acquired time-dependent response signal with respect to the target subject, and a first profile obtained from a time-dependent response signal with respect to a subject derived from a target with a favorable health condition and a second profile obtained from a time-dependent response signal with respect to a subject derived from a target with an unfavorable health condition.

### REFERENCE TO RELATED APPLICATIONS

This application claims priority from Japanese Patent Application No. 2024-134679 filed on Aug. 9, 2024. The entire content of the priority application is incorporated herein by reference.

### BACKGROUND ART

The present invention relates to a determination method, a determination device, and a recording medium.

An olfactory sensor has been proposed as an artificial device that is used as a substitute for the olfactory sense in five human senses. For example, in Japanese Patent Application Laid-Open No. 2018-113957, a low-cost odor sensor including a transistor including a gate electrode containing aluminum or aluminum oxide, an insect cell having an olfactory receptor arranged on a gate electrode, and a detection device detecting a current generated by a transistor when the insect cell reacts with an odor is disclosed.

### SUMMARY

However, in the technology disclosed in Japanese Patent Application Laid-Open No. 2018-113957, a health condition is not determined by using the olfactory sensor.

An object of the present disclosure is to provide a determination method and the like, in which the health condition of a target can be determined by using an olfactory sensor.

A determination method according to one aspect of the disclosure causing a computer to execute processing of: acquiring a time-dependent response signal with respect to a target subject derived from a determination target, detected by using an olfactory sensor including cells that have olfactory receptors and respond to odorous molecules; and determining a health condition of the determination target, on the basis of a profile of the acquired time-dependent response signal with respect to the target subject, and a first profile obtained from a time-dependent response signal with respect to a subject derived from a target with a favorable health condition and a second profile obtained from a time-dependent response signal with respect to a subject derived from a target with an unfavorable health condition.

A determination device according to one aspect of the disclosure, includes a control unit that executes processing of: acquiring a time-dependent response signal with respect to a target subject derived from a determination target, detected by using an olfactory sensor including cells that have olfactory receptors and respond to odorous molecules; and determining a health condition of the determination target, on the basis of a profile of the acquired time-dependent response signal with respect to the target subject, and a first profile obtained from a time-dependent response signal with respect to a subject derived from a target with a favorable health condition and a second profile obtained from a time-dependent response signal with respect to a subject derived from a target with an unfavorable health condition.

A computer program recorded in a recording medium according to one aspect of the disclosure, causes a computer to execute processing of: acquiring a time-dependent response signal with respect to a target subject derived from a determination target, detected by using an olfactory sensor including cells that have olfactory receptors and respond to odorous molecules; and determining a health condition of the determination target, on the basis of a profile of the acquired time-dependent response signal with respect to the target subject, and a first profile obtained from a time-dependent response signal with respect to a subject derived from a target with a favorable health condition and a second profile obtained from a time-dependent response signal with respect to a subject derived from a target with an unfavorable health condition.

According to the disclosure, it is possible to determine the health condition of the target by using the olfactory sensor.

The above and further objects and features will more fully be apparent from the following detailed description with accompanying drawings

### BRIEF DESCRIPTION OF DRAWINGS

FIG. 1 is an outline diagram of a determination system.

FIG. 2 is a schematic view illustrating a configuration example of an olfactory sensor.

FIG. 3 is a block diagram illustrating a configuration of a determination device.

FIG. 4 is a diagram illustrating a content example of information stored in a detection DB.

FIG. 5 is a block diagram illustrating a configuration of a terminal device.

FIG. 6 is a diagram illustrating an example of a time-dependent change in luminescence intensity of a sensor cell.

FIG. 7 is a schematic view illustrating an example of a reception screen.

FIG. 8A is a diagram illustrating an example of a reference profile according to a cell that is selected for disease determination.

FIG. 8B is a diagram illustrating an example of a reference profile according to a cell that is not selected for disease determination.

FIG. 9 is a schematic view illustrating an example of a result screen showing a determination result.

FIG. 10 is a schematic view illustrating another example of the result screen showing the determination result.

FIG. 11 is a flowchart illustrating an example of a processing procedure for generating a reference profile.

FIG. 12 is a flowchart illustrating an example of a processing procedure for determining a possibility of a disease.

FIG. 13 is a flowchart illustrating an example of a processing procedure executed by a determination device of a second embodiment.

FIG. 14A is a diagram illustrating an example of a response profile according to the presence or absence of correction processing.

FIG. 14B is a diagram illustrating an example of a response profile according to the presence or absence of correction processing.

FIG. 15 is a block diagram illustrating a configuration example of a determination device of a third embodiment.

FIG. 16 is an explanatory diagram illustrating an outline of a learning model.

FIG. 17 is a flowchart illustrating an example of a processing procedure executed by a determination system of the third embodiment.

### DESCRIPTION

The disclosure will be described in detail with reference to the drawings illustrating embodiments thereof.

### First Embodiment

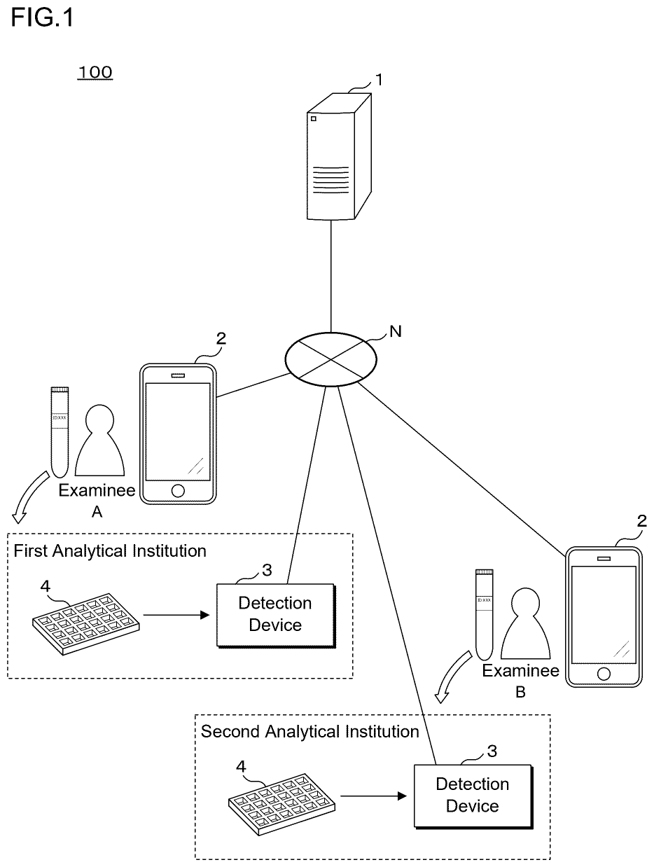

FIG. 1 is an outline diagram of a determination system 100. The determination system 100 includes a determination device 1, a terminal device 2, a detection device 3, and an olfactory sensor 4. The determination device 1 is connected to the terminal device 2 and the detection device 3 via a network N such as the internet such that communication is available. The determination system 100 provides a service in which the health condition of a target is determined on the basis of a detection result of odorous molecules in a subject derived from an examinee detected by the detection device 3 and the olfactory sensor 4, and the determination device 1 presents a determination result via the terminal device 2.

In the following embodiment, a case of determining the possibility of a disease in the examinee, on the basis of odorous molecules detected from the urine of the examinee, will be described as an example. The type of disease to be a determination target is not limited, and examples thereof include an infectious disease such as influenza or COVID-19, cancer, a lifestyle disease such as type 2 diabetes, a cardiac disease, or a cerebrovascular disease, a neurological disease such as a Parkinson's disease or an Alzheimer's disease, and the like. In a case where the disease is a cancer, for example, the disease may be various cancers such as a lung cancer, an esophageal cancer, a breast cancer, a gastric cancer, a liver cancer, a pancreatic cancer, a gallbladder cancer, a biliary cancer, a colorectal cancer, a kidney cancer, a bladder cancer, an ovarian cancer, a uterine cancer, a prostate cancer, an oral cancer, and a pharyngeal cancer.

The determination device 1 is an information processing device that is capable of performing various types of information processing, and the transmission and reception of information, and for example, is a server computer, a personal computer, a quantum computer, or the like. The determination device 1 attains a service in which a signal indicating the response of the odor with respect to the urine of the examinee is acquired, the possibility of the disease in the examinee is determined on the basis of the acquired signal, and the determination result is provided to a user via the terminal device 2. The determination device 1 may be a local computer provided in a facility in which the detection device 3 is installed.

The terminal device 2 is an information processing terminal that is used by the examinee, and for example, is a personal computer, a smart phone, a tablet terminal, or the like. The terminal device 2 displays the determination result received from the determination device 1. The examinee is an example of the user who receives the provision of the determination result. The determination result is not limited to being provided to the examinee, and for example, may be provided to medical personnel, a person in charge of an analytical institution performing analysis, or the like. The number of terminal devices 2 connected to the determination device 1 may be 1 or 3 or more.

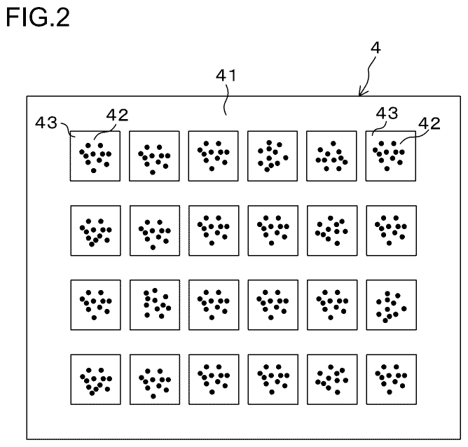

The olfactory sensor 4 and the detection device 3, for example, are managed by an analytical institution carrying out an analysis operation of the subject. The olfactory sensor 4 includes cells 42 (refer to FIG. 2) that express olfactory receptors as a detection element, and outputs a signal indicating the response of the olfactory receptor with respect to the odorous molecules. The detection device 3 detects a response signal of the olfactory sensor 4 in a time-dependent manner. The detection device 3 has not only a function as a detector carrying out the detection of the response signal described above, but also a function as a computer performing various types of data processing or communication with an external device, and transmits detection data of the response signal obtained by the detection to the determination device 1 via the network N. Note that the detector and the computer may be provided separately, and may be configured such that communication is available. The number of detection devices 3 connected to the determination device 1 may be 1 or 3 or more.

FIG. 2 is a schematic view illustrating a configuration example of the olfactory sensor 4. FIG. 2 is a diagram when the olfactory sensor 4 is seen from the above. The olfactory sensor 4 includes a substrate 41, wells 43 formed on the substrate 41, and cells 42 arranged in the well 43. As the substrate 41, for example, a “384 well plate” on which 384 wells 43 are formed is used. In FIG. 2, for simplicity of illustration, as the substrate 41, a “24 well plate” on which 24 wells 43 are formed is illustrated. In this embodiment, the cells 42 including a plurality of cells with the same type are arranged in each of a plurality of wells 43. That is, a plurality of cells with the same type are densely seeded in each of the wells 43. Note that the cell 42 may include one cell. Each of the cells has an olfactory receptor. The cell 42 functions as a sensor cell outputting a signal indicating the response of the olfactory receptor with respect to the odorous molecules. The olfactory receptor responds to specific odorous molecules. By detecting the response signal based on a combination between the olfactory receptor provided in the olfactory sensor 4 and the odorous molecules, it is possible to quantitatively detect the odor contained in the urine of the examinee.

The substrate 41, for example, is composed of materials such as glass, silicon, ceramics, a resin, and a metal. The surface of the substrate 41 may be subjected to, for example, a surface treatment such as a plasma treatment, a corona treatment, and a UV-Ozone treatment, or coating using polypeptide or the like. The substrate 41 may be in a shape in which the odor can be detected by using the cells 42 arranged on the substrate 41, and for example, may be in the shape of a rectangular plate. The size of the substrate 41 is not particularly limited, and can be suitably set in accordance with the number of cells 42 arranged on the substrate 41, or the like.

As the olfactory receptor, an olfactory receptor derived from an animal can be used. Examples of the animal include insects, vertebrates, mammals, and the like, and for example, olfactory receptors of flies, mosquitoes, mice, rats, rabbits, cattle, dogs, humans, and the like can be used. As the olfactory receptor, an insect olfactory receptor is preferable. The insect olfactory receptor is an ion channel receptor, and in a case where the insect olfactory receptor is combined with the odorous molecules, an olfactory receptor complex in which the olfactory receptor and an olfactory receptor co-receptor are formed is activated, and cations flow into the cell.

The amino acid sequence and the coding sequence of the olfactory receptor and the olfactory receptor co-receptor are known, or can be easily identified by sequence identity search based on a known sequence. Further, an amino acid mutation with respect to a known amino acid sequence can be included. The amino acid mutation, for example, is the substitution, the insertion, the addition, or the deletion of an amino acid.

As the cell 42, a specific cell that naturally expresses an olfactory receptor may be used, or a genetically modified cell implanted with a gene of an olfactory receptor may be used. The genetically modified cell can be prepared by the genetic transformation of cells using a vector implanted with an olfactory receptor gene. In a case where the olfactory receptor is an insect olfactory receptor, it is more preferable to implant the olfactory receptor with a gene of an olfactory receptor co-receptor.

Further, the cell 42 may have a fluorescent protein or a luminescent protein. In the cell 42, in a case where odorous molecules are combined with ion channel olfactory receptors, cations such as calcium ions flow into the cell. By introducing a gene expressing a fluorescent protein of which the fluorescence intensity is changed in accordance with an ion concentration or a luminescent protein of which the luminescence intensity is changed into the cell 42, it is possible to detect the response of the cell with respect to the odorous molecules by a change in the fluorescence intensity or the luminescence intensity. That is, it is possible to detect the odorous molecules by a change in the fluorescence intensity or the luminescence intensity. Examples of such proteins include aequorin, Yellow Cameleon, GCaMP, and the like.

A calcium ion-sensitive fluorescent dye may be introduced into the cell 42. By introducing the calcium ion-sensitive fluorescent dye into the cell, it is possible to detect the inflow of the calcium ions into the cell due to the combination between the odorous molecules and the olfactory receptors by a change in the fluorescence intensity. Examples of such a calcium ion-sensitive fluorescent dye include Fura-2, Fluo-3, Fluo-4, and the like.

The detection device 3 detects a signal indicating the response of the cell 42 based on the combination between the olfactory receptors and the odorous molecules. The detection device 3, for example, detects the luminescence of each of the wells 43 in the olfactory sensor 4, as a detection target. The detection device 3, for example, includes a photomultiplier tube, and detects fluorescence or luminescence based on a change in the ion concentration in the cell. The response signal of the cell 42 detected by the detection device 3 is not limited to the fluorescence intensity or the luminescence intensity, and may be an electrical signal (a potential) based on a change in the ion concentration in the cell. The response signal may be a moving image or a still image obtained by capturing the state of the luminescence of the cell 42 with an imaging device such as a CCD camera.

As illustrated in FIG. 2, the olfactory sensor 4 of this embodiment includes a plurality of cells 42 arranged on the substrate 41. In the example illustrated in FIG. 2, the plurality of cells 42 are vertically and horizontally arranged at regular intervals on the upper surface of the substrate 41.

In general, the olfactory receptor has selectivity with respect to the odorous molecules. Accordingly, the plurality of cells 42 expressing different olfactory receptors are arranged on the substrate 41, and the response of each of the cells 42 is detected, and thus, the plurality of types of odors can be detected. One cell 42 may have one type of olfactory receptor, or may be a plurality of types of olfactory receptors. The olfactory sensor 4 may include a plurality of same cells 42. The olfactory sensor 4 of this embodiment includes different types of cells 42, and each of the cells 42 has one type of olfactory receptor.

The number, the type, and the arranged position of olfactory receptors used in the olfactory sensor 4 can be suitably set in accordance with the type of odorous molecules to be detected and the type of disease to be a determination target. The olfactory receptors may be a specific combination with respect to the odorous molecules to be detected, or may be used by comprehensively combining a plurality of olfactory receptors with various odorous molecules. For example, the number of cells 42 mounted on one olfactory sensor 4, that is, the total number of sensor cells mounted on one olfactory sensor 4 can be 1 or more and 2000 or less, and the number of types of cell 42 mounted on one olfactory sensor 4 can be 1 or more and 2000 or less.

A method for detecting the response signal is not limited to the examples described above, and a suitable method can be used in accordance with the type of olfactory receptor in the olfactory sensor 4 or the type of odorous molecules sensed by the olfactory receptor.

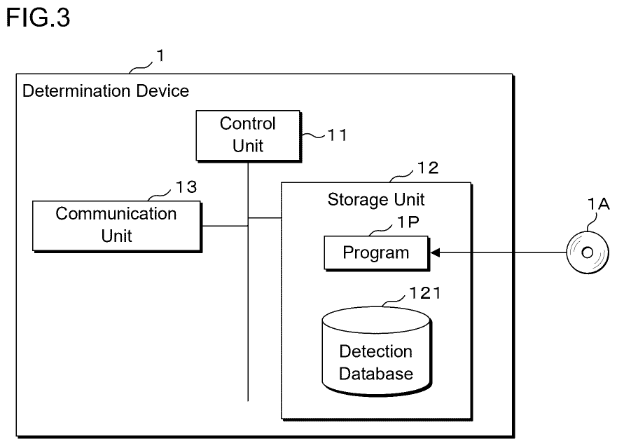

FIG. 3 is a block diagram illustrating the configuration of the determination device 1. The determination device 1 includes a control unit 11, a storage unit 12, and a communication unit 13. The determination device 1 may be a single computer, or may be a computer system composed of a plurality of computers, a peripheral device, and the like. The determination device 1 may be a virtual machine in which the substance is virtualized, or may be a cloud.

The control unit 11 includes one or a plurality of arithmetic processing devices such as a central processing units (CPU) and a graphics processing unit (GPU). The control unit 11 controls each constituent unit and executes processing by using a built-in memory such as a read only memory (ROM) or a random access memory (RAM), a clock, a counter, and the like. The function unit of the control unit 11 may be attained by software, or a part or all thereof, for example, may be attained by hardware such as an application specific integrated circuit (ASIC) and a field programmable gate array (FPGA).

The storage unit 12, for example, includes a non-volatile memory such as a hard disk, a flash memory, and a solid state drive (SSD). The storage unit 12 may be one or a plurality of external storage devices that are separated from the determination device 1 and externally connected to the determination device. The storage unit 12 stores various computer programs and data that the control unit 11 refers to. The storage unit 12 stores a program 1P causing a computer to execute processing relevant to the determination of the possibility of a disease, and a detection database (DB) 121. Further, a reference profile described below may be stored in the storage unit 12.

The computer program (a program product) including the program 1P may be provided by a non-transitory recording medium 1A in which the computer program is recorded to be readable. The storage unit 12 stores the computer program read out from the recording medium 1A by a reading device, which is not illustrated. The recording medium 1A, for example, is a magnetic disk, an optical disk, a semiconductor memory, or the like. In addition, the computer program may be downloaded from an external server connected to a communication network, and may be stored in the storage unit 12. The program 1P may be a single computer program, or may be composed of a plurality of computer programs. In addition, the program 1P may be executed on a single computer, or may be executed by a plurality of computers in cooperation.

The communication unit 13 includes a communication device that attains communication via the network N. The control unit 11 transmits and receives data between the terminal device 2 and the detection device 3 via the communication unit 13.

The configuration of the determination device 1 not limited to the examples described above, and for example, may include a manipulation unit for receiving the manipulation of the user, a display unit displaying an image, and the like.

The determination device 1 and the detection device 3 are not limited to transmitting and receiving data via the network N. The determination device 1, for example, may include an input interface for connecting the detection device 3, and may receive data output from the detection device 3 via a signal line or the like.

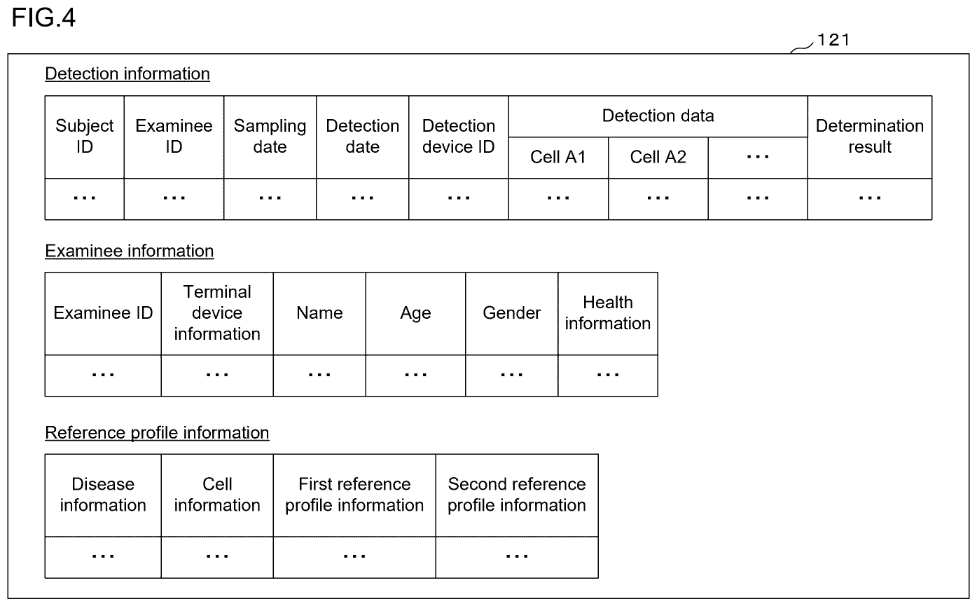

FIG. 4 is a diagram illustrating a content example of information stored in detection DB 121. The detection DB 121 is a database in which detection information relevant to each of a plurality of subjects, examinee information relevant to the examinee, and reference profile information relevant to the reference profile are stored.

In a detection information table, for example, a record is stored in which information such as an examinee ID, a sampling date, a detection device ID, a detection date, detection data, and a determination result is associated with a subject ID as a key. The subject ID is identification information for uniquely specifying the subject sampled from the examinee. The subject ID may be an ID given to a storage container storing a specific subject. The examinee ID is identification information for identifying the examinee. The sampling date represents date and time when the subject is sampled. The detection device ID is identification information for identifying the detection device 3 used to detect the response signal with respect to the subject. The detection date represents date and time when the response signal with respect to the subject is detected by the detection device 3.

The detection data includes information indicating the response signal with respect to the subject. The detection data, for example, is the profile of the response signal detected time-dependently. In this embodiment, the profile of the response signal as the detection data is time-dependent data of the luminescence intensity. The detection data is generated for each of the cells 42 in the olfactory sensor 4. The determination result represents the possibility of a disease based on the detection data. The determination result, for example, may be indicated by the presence or absence of the possibility, or may be indicated by the degree of possibility classified into a plurality of stages. The information of the detection information table, for example, is collected via the detection device 3.

In an examinee information table, for example, a record is stored in which information such as terminal device information, and attribute information of the examinee is associated with the examinee ID as a key. The detection information table and the examinee information table are associated with each other by the examinee ID. The terminal device information is information for identifying the terminal device 2 that is used by the examinee, and for example, includes an address indicating an output destination of the determination result, a device ID, and the like. The attribute information, for example, includes the name, the age, the gender, the health information, or the like of the examinee. The health information may include information such as information relevant to the health condition of the examinee, a current symptom, a medical history, an examination result, and a health diagnosis result. The information of the examinee information table, for example, is collected via the terminal device 2.

In a reference profile information table, for example, a record is stored in which information such as cell information, and first reference profile information and second reference profile information is associated with a disease ID as a key. The disease information is information for identifying the disease to be the determination target, and for example, includes the disease ID, the name of disease, and the like. The cell information is information for identifying the cell 42 in the olfactory sensor 4, and is information representing the type of cell selected for disease determination. The first reference profile information and the second reference profile information are information relevant to a first reference profile and a second reference profile corresponding to the cell information. The details of the first reference profile and the second reference profile will be described below. Note that FIG. 4 is an example, and the contents of the information stored in the detection DB 121 may not be limited. In addition, the method for storing the data illustrated in FIG. 4 is an example, and other storage forms may be available insofar as the contents of the data and the relationship between the data pieces are maintained.

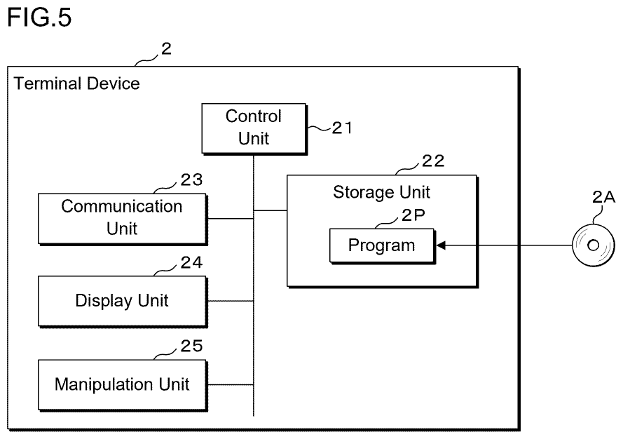

FIG. 5 is a block diagram illustrating the configuration of the terminal device 2. The terminal device 2 includes a control unit 21, a storage unit 22, a communication unit 23, a display unit 24, and a manipulation unit 25.

The control unit 21 includes one or a plurality of arithmetic processing devices such as a CPU, a MPU, and a GPU. The control unit 11 controls each constituent unit and executes processing by using a built-in memory such as a ROM or a RAM, a clock, a counter, and the like.

The storage unit 22, for example, includes a non-volatile memory such as a hard disk, a flash memory, and a SSD. The storage unit 22 stores various computer programs and data that the control unit 21 refers to. The storage unit 22 stores a program 2P causing a computer to execute processing relevant to the acquisition of the determination result of the possibility of the disease.

The computer program (a computer program product) including the program 2P may be provided by a non-transitory recording medium 2A in which the computer program is recorded to be readable. The storage unit 22 stores the computer program read out from the recording medium 2A by a reading device, which is not illustrated. The recording medium 2A, for example, is a magnetic disk, an optical disk, a semiconductor memory, or the like. In addition, the computer program may be downloaded from an external server connected to a communication network, and may be stored in the storage unit 22. The program 2P may be a single computer program, or may be composed of a plurality of computer programs. In addition, the program 2P may be executed on a single computer, or may be executed by a plurality of computers in cooperation.

The communication unit 23 includes a communication device that attains communication via the network N. The control unit 21 transmits and receives data with respect to the determination device 1 via the communication unit 23.

The display unit 24, for example, includes a display device such as a liquid crystal display and an organic electro luminescence (EL) display. The display unit 24 displays various types of information including the determination result, in accordance with an instruction from the control unit 21.

The manipulation unit 25 is an interface that receives the manipulation of the user. The manipulation unit 25, for example, includes a keyboard, a mouse, a touch panel device with a built-in display, a speaker, a microphone, and the like. The manipulation unit 25 receives manipulation input from the user, and sends a control signal according to manipulation contents to the control unit 21.

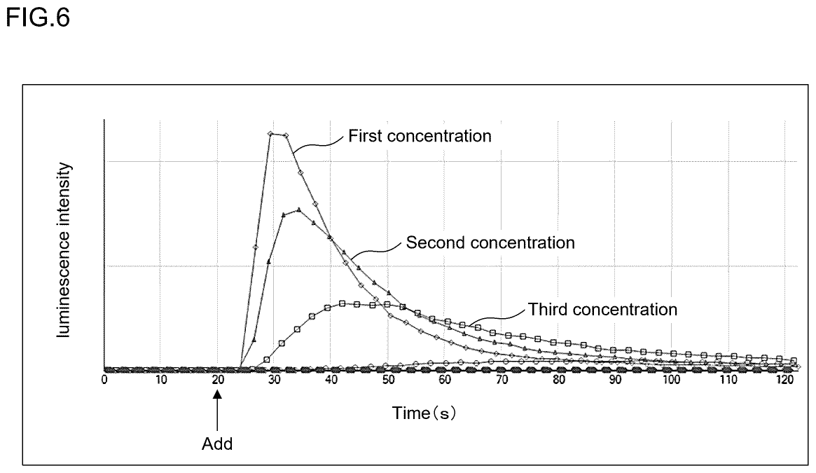

FIG. 6 is a diagram illustrating an example of a time-dependent change in the luminescence intensity of the sensor cell. In a graph illustrated in FIG. 6, a vertical axis is the luminescence intensity, and a horizontal axis is an elapsed time (s). In FIG. 6, a detection result of the luminescence intensity in a case where a solution containing specific odorous molecules is added to cells having olfactory receptors that are combined with the odorous molecules is shown for each concentration of the odorous molecules. In FIG. 6, a first concentration, a second concentration, and a third concentration are set in descending order of the concentration of the odorous molecules.

As illustrated in FIG. 6, a profile indicating a time-dependent change in the luminescence intensity is concentration-dependently changed. In this embodiment, by estimating the property of an odorous molecular group in specific urine that can be detected by the sensor cell, on the basis of a response profile of the luminescence intensity detected from a urine sample of the examinee, the possibility of the disease in the examinee is determined.

Hereinafter, a determination processing flow in the determination system 100 of this embodiment will be described with specific examples.

The determination device 1 receives an application for a determination service from the examinee. The user (the examinee) who desires to use the service makes an application via a reception screen 50 provided from the determination device 1, for example, by using the terminal device 2.

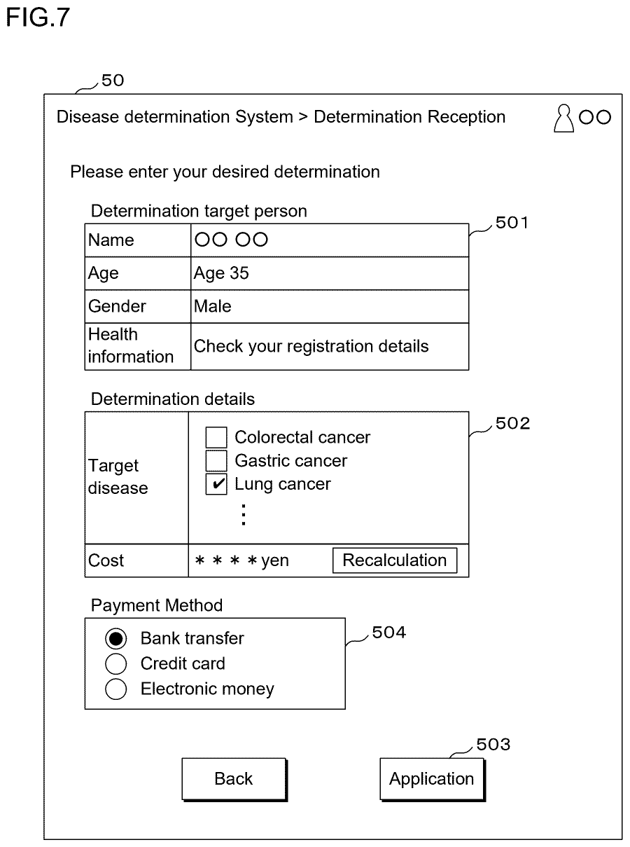

FIG. 7 is a schematic view illustrating an example of the reception screen 50. In a case where a request for login and the reception screen using account information according to the manipulation of the examinee is received via the terminal device 2, as illustrated in FIG. 7, the determination device 1 outputs the reception screen 50 to the terminal device 2 to be displayed on the display unit 24.

The reception screen 50 includes an examinee information reception section 501 for receiving the input of the examinee information relevant to the examinee to be the determination target, and a disease information reception section 502 for receiving the input of a target disease desired to be determined. The examinee inputs the name of the examinee to be the determination target, or the like to the examinee information reception section 501 by using the manipulation unit 25. Note that in a case where the examinee is registered in advance as a user, the examinee information according to the logged-in account information may be read out with reference to the detection DB 121, and the read examinee information may be displayed in the examinee information reception section 501.

The disease information reception section 502 displays a plurality of diseases that can be determined by the determination system 100 to be selectable.

The examinee is capable of inputting the designation of one or a plurality of diseases desired to be determined, for example, by selecting a checkbox associated with each disease. In the example illustrated in FIG. 7, a lung cancer is selected as the target disease.

In a case where an application button 503 for designating the application for the determination is selected in a state where various types of information is input to the examinee information reception section 501 and the disease information reception section 502, the terminal device 2 transmits the received examinee information and target disease to the determination device 1. The determination device 1 receives the examinee information and the target disease, and receives the application for the determination. The determination device 1 stores the received examinee information and target disease in the storage unit 12, and as necessary, transmits information according to the examinee information and the target disease to the detection device 3 of the analytical institution that carries out the examination.

In addition, as illustrated in FIG. 7, the reception screen 50 may include a payment method reception section 504. The examinee is capable of designating a desired payment method by selecting a specific payment method from the payment method reception section 504. The terminal device 2 may execute processing for online payment or credit card payment with respect to a predetermined payment server or the like, in accordance with the received payment method.

Note that information relevant to the application for the determination service is not limited to being received via the terminal device 2, and the determination device 1, for example, may acquire the examinee information or the like by receiving input from the user.

In a case where the application is completed, a urine sample container is sent to the examinee. The urine sample container may be distributed via a predetermined store, analytical institution, or the like. The urine sample container, for example, is attached with a label on which a two-dimensional code or a three-dimensional code representing the examinee ID is printed. The examinee stores the sampled urine in the urine sample container, and submits the urine sample container to the analytical institution. The urine sample container may be submitted with the attribute information of the examinee such as the name of the examinee, the sampling date, an application number issued when the determination application is made, or the like.

In the analytical institution, the urine sample container is received. The detection device 3, for example, acquires the subject ID of the subject to be an examination target, the examinee ID, the sampling date, and the like by receiving input from the person in charge.

Next, in the analytical institution, the response signal is detected by using the olfactory sensor 4. In the olfactory sensor 4, a predetermined number of cells 42 each having different olfactory receptors are arranged into the shape of an array. The olfactory sensor 4, for example, is prepared by implanting chromosomal genome DNA of each cell with DNA including a specific insect olfactory receptor coding sequence, a specific insect olfactory receptor co-receptor coding sequence, and a calcium-sensitive luminescent protein coding sequence, which are arranged under the control of a promoter sequence.

By bringing a predetermined amount of urine sample derived from the examinee into contact with the olfactory sensor 4, the luminescence intensity for each cell is time-dependently detected by the detection device 3. Accordingly, the response profile representing a time-dependent change in the luminescence intensity is acquired. The response profile is generated for each of the cells 42 in the olfactory sensor 4. The detection device 3 associates the subject ID of the subject to be the examination target, the examinee ID, the sampling date, the detection device ID, the detection date, and the like with the acquired response profile, and transmits the response profile to the determination device 1.

The determination device 1 may perform predetermined preprocessing on the detection data received from the detection device 3 to generate the response profile. The measurement of the luminescence intensity with respect to the olfactory sensor 4 may be collectively executed on a plurality of sensor cells, which are arrayed. In such a case, it is assumed that the raw detection data output from the detection device 3 is data including the luminescence intensity with respect to the plurality of sensor cells at various times (detection times). Alternatively, detection values according to urine samples derived from plurality of examinees may be mixed into the detection data. The determination device 1 sorts the raw detection data including the plurality of detection values for each sensor cell or each urine sample derived from the examinee, time-dependently arranges the luminescence intensity, and converts the luminescence intensity into a predetermined data format to generate the response profile. In a case where the time of the luminescence intensity in the raw detection data is different, the determination device 1 may interpolate data by using a predetermined interpolation method to standardize the time of each type of data in the response profile.

The determination device 1 determines the possibility of a disease in the examinee, on the basis of the obtained response profile of the urine sample derived from the examinee. In this embodiment, by comparing the response profile of the urine sample derived from the examinee to be an analysis target with the reference profile generated in advance, the determination of the possibility of a disease is performed.

The reference profile includes first reference profile based on luminescence intensity detected from urine derived from an examinee with a favorable health condition, and the second reference profile based on luminescence intensity detected from a urine sample derived from an examinee with an unfavorable health condition. When a good health condition is referred to as a first health condition, a bad health condition corresponds to a second health condition, which is worse than the first health condition. As an example, in a case where the target disease is a lung cancer, the examinee with a favorable health condition indicates a non-cancer patient who is not suffering from a cancer, the examinee with an unfavorable health condition indicates a lung cancer patient who is suffering from a lung cancer. The reference profile, for example, is generated in advance and stored in the storage unit 12.

The first reference profile and the second reference profile is obtained by detecting the odorous molecules described above with the olfactory sensor 4 by using urine sampled from the non-cancer patient (hereinafter, also referred to as normal urine) and urine sampled from the lung cancer patient (hereinafter, also referred to as lung cancer urine), and generating the response profile. The first reference profile and the second reference profile are prepared for each of the cells 42.

It is preferable that the first reference profile is generated on the basis of response profiles of a plurality of types of normal urine obtained from a plurality of non-cancer patients. Similarly, it is preferable that the second reference profile is generated on the basis of response profiles of a plurality of types of lung cancer urine obtained from a plurality of lung cancer patients. For example, the statistical value of the luminescence intensity detected from each type of normal urine is calculated for each elapsed time, and the first reference profile is generated on the basis of the obtained time-dependent statistical value. As the statistical value, a mean value or a median value is preferable, a weighted mean, a geometric mean, or a median value is more preferable, and a geometric mean is most preferable. By the same method, the second reference profile is generated on the basis of the response profiles of the plurality of types of lung cancer urine obtained from the plurality of lung cancer patients.

Further, one or a plurality of cells 42 suitable for the determination of the lung cancer as a disease to be determined are selected from a plurality of types of cells 42 included in the olfactory sensor 4. The cell suitable for the determination of the lung cancer is a cell indicating reactivity according to the lung cancer urine. The cell suitable for the determination of the lung cancer is preferably a cell having reactivity that is significantly changed in accordance with the presence or absence of the lung cancer and having a significant difference in the response profile. The cell suitable for the determination of the lung cancer may be a cell that reacts with odorous molecules contained comparatively more in the lung cancer urine than in the normal urine, or may be a cell that reacts with odorous molecules contained comparatively more in the normal urine than in the lung cancer urine. In this embodiment, the cell 42 that reacts with the odorous molecules contained more in the lung cancer urine than in the normal urine is the cell 42 suitable for the determination of the lung cancer.

In a state of generating the olfactory sensor 4, it is not easy to specify in advance the cell 42 suitable for the determination of a disease to be determined. Therefore, in this embodiment, the olfactory sensor 4 on which the plurality of types of cells 42 are mounted is prepared, and the cell 42 suitable for the determination of the disease is specified from the plurality of types of cells 42 for each disease to be determined.

A method for specifying the cell 42 suitable for the determination of the disease is not limited, and for example, the cell 42 can be specified on the basis of the degree of dissimilarity between the first reference profile and the second reference profile. The determination device 1 may calculate the degree of dissimilarity between the first reference profile and the second reference profile, and may specify the cell 42 in which the calculated degree of dissimilarity is greater than or equal to a first threshold value set in advance, as the cell 42 for disease determination.

As the degree of dissimilarity between the first reference profile and the second reference profile, for example, a difference value between the maximum value of the luminescence intensity of the first reference profile and the maximum value of the luminescence intensity of the second reference profile is used. As the degree of dissimilarity, an area value surrounding the first reference profile and the second reference profile (an intensity difference integral value obtained by integrating an intensity difference between the first reference profile and the second reference profile) may be used. As a difference in the maximum value of the intensity between the reference profiles or the intensity difference integral value increases, the similarity between the first reference profile and the second reference profile decreases, and a change in the reactivity according to the presence or absence of the disease increases. That is, it is considered that a response difference of the cell 42 according to the presence or absence of the disease increases, and availability in the disease determination increases. Note that the degree of dissimilarity between the first reference profile and the second reference profile is used in the above, but the degree of similarity may be used.

The cell 42 for disease determination may be selected in consideration of disease determination accuracy using the cell 42. For example, for the plurality of cells 42 primarily selected for the disease determination, response profiles with respect to a plurality of samples for examination are acquired, and the possibility of the disease is determined in accordance with the following determination method by using the acquired response profiles. The determination accuracy when using each of the cells 42 primarily selected on the basis of the determination result according to each of the cells 42, and the property of a well-known sample for examination (the normal urine or the lung cancer urine) is obtained. The determination accuracy, for example, includes AUC of a ROC curve, a recall rate, specificity, a correctness rate, accuracy, and the like. The type and the number of cells 42 selected for the disease determination are selected such that each determination accuracy is optimized (for example, each determination accuracy is maximized) on the basis of the value of the determination accuracy. The cell 42 for disease determination is secondarily selected in accordance with the type and the number of specified cells 42.

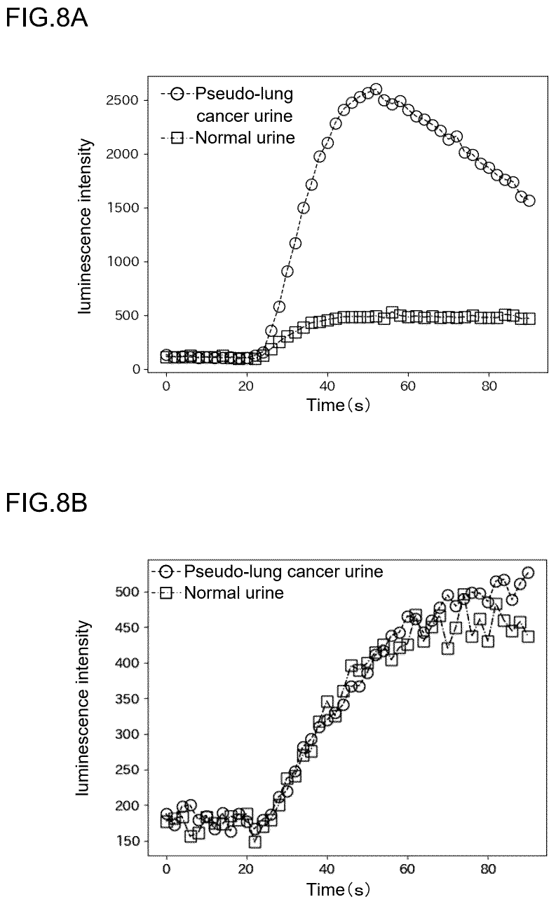

FIG. 8 is a diagram illustrating an example of a reference profile according to a cell that is selected for the disease determination and a reference profile according to a cell that is not selected for the disease determination. FIG. 8A illustrates an example of the reference profile according to the cell that is selected for the disease determination, and FIG. 8B illustrates an example of the reference profile according to the cell that is not selected for the disease determination. In the case of the cell that is for the disease determination, the shape of the response profile is significantly different between the normal urine and pseudo-lung cancer urine (urine obtained by adding odorous molecules derived from a lung cancer to the normal urine). On the other hand, in the case of the cell that is not for the disease determination, the response profile is not significantly different between the normal urine and the pseudo-lung cancer urine.

Note that in a case where the type of cell 42 suitable for the determination of a specific disease is well-known, a step of selecting the cell 42 for disease determination may be omitted.

The determination device 1 determines the degree of similarity between the response profile of the urine sample of the examinee corresponding to the selected cell 42 for disease determination, and each of the first reference profile and the second reference profile corresponding to the cell 42 for disease determination to determine the possibility of a lung cancer. In a case where the degree of similarity with the first reference profile is higher than the degree of similarity with the second reference profile, it is determined that there is no possibility of the lung cancer. In a case where the degree of similarity with the second reference profile is higher than the degree of similarity with the first reference profile, it is determined that there is the possibility of the lung cancer. The determination device 1 may determine the possibility of the lung cancer in a plurality of stages, a percentage, or the like, in accordance with the value of the degree of similarity.

A method for determining the degree of similarity is not limited, and for example, the absolute value of a difference between the luminescence intensity of the response profile of the urine sample of the examinee and the luminescence intensity of the reference profile may be calculated for each elapsed time, and a sum of the calculated absolute values of the differences may be set as a determination index of the degree of similarity. As the sum of the absolute values of the differences in the luminescence intensity decreases, the degree of similarity increases.

In a case where a plurality of cells 42 for disease determination are selected, the determination device 1 individually determines the possibility of the lung cancer based on each of the cells 42 for disease determination, and integrates the individually determined possibilities of the lung cancer to comprehensively determine the possibility of the lung cancer. The determination device 1, for example, comprehensively determines the majority rule of the determination result based on each of the cells 42. In the comprehensive determination, weighting may be performed to increase the weight of the determination result based on the specific cell 42. The cell 42 on which the weighting is performed may be a cell 42 with higher availability in the disease determination, a cell 42 with a higher degree of similarity, a cell 42 with a small variation in the response signal, and the like.

The determination of the possibility of the lung cancer may be performed by using a machine learning method. The determination device 1 prepares in advance a determination model that outputs the possibility of the lung cancer when the degree of similarity between the response profile of each of the cells 42 for disease determination and the reference profile is input. The degree of similarity includes at least one of the degree of similarity between the response profile of the urine sample of the examinee and the first reference profile, and the degree of similarity between the response profile and the second reference profile.

The determination device 1 inputs the degree of similarity relevant to the response profile of each of the cells 42 for disease determination to the determination model, and acquires the possibility of the lung cancer output from the determination model. The type of cell 42 may be input to the determination model along with the degree of similarity. The determination result of the possibility of the lung cancer output from the determination model corresponds to a comprehensive determination result. In this case, the individual determination based on each of the cells 42 may be omitted.

Note that in a case where the type of cell 42 suitable for the determination of a specific disease is well-known, for each type of disease to be determined, the olfactory sensor 4 may be individually generated by using only the cell 42 suitable for the determination of the disease. In this case, the luminescence intensity is detected by using the individual olfactory sensor 4 according to the selected target disease, and the possibility of the disease is determined on the basis of the detection result of each of the cells 42 in the individual olfactory sensor 4. The determination device 1 stores a series of obtained information, for example, the subject ID, the examinee ID, the sampling date, the detection device ID, the detection date, the response profile, the determination result, and the like in association with each other, in the detection DB 121. In addition, the determination device 1 outputs the determination result to the terminal device 2 of the examinee.

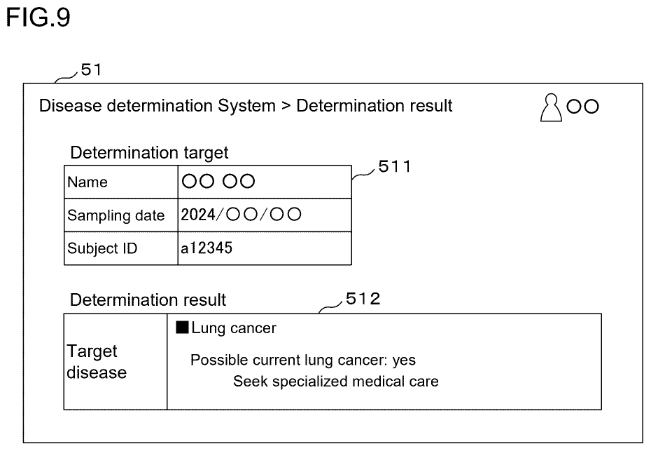

FIG. 9 is a schematic view illustrating an example of a result screen 51 showing the determination result. The result screen 51 includes a first display portion 511 for displaying information relevant to the subject to be the determination target, and a second display portion 512 for displaying the determination result.

The determination device 1 displays the information relevant to the examinee and the subject to be the determination target in the first display portion 511, on the basis of the information stored in the detection DB 121. In the first display portion 511, for example, the name of the examinee, the sampling date of the subject, and the subject ID are displayed.

In addition, the determination device 1 displays the determination result indicating the presence of absence of the possibility of the disease in the second display portion 512. In a case where determination results with respect to a plurality of types of diseases are acquired, the result screen 51 may include a plurality of second display portions 512 corresponding to each target disease. Each of the second display portions 512 displays the target disease, and the determination result according to the target disease.

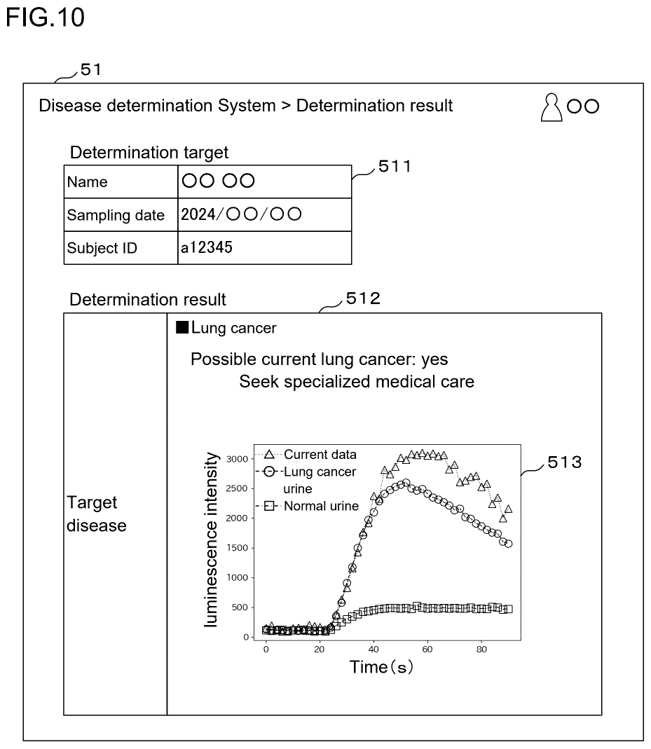

FIG. 10 is a schematic view illustrating another example of the result screen 51 showing the determination result. In the example illustrated in FIG. 10, the second display portion 512 of the result screen 51 further includes a detection result display section 513 for displaying the detection result with respect to the subject detected by the olfactory sensor 4. In the detection result display section 513, the response profile corresponding to the subject is displayed.

In the detection result display section 513, a determination reference using disease determination processing, that is, the first reference profile and the second reference profile may be displayed, in addition to the response profile corresponding to the subject. In FIG. 10, for simplicity of description, only one type of response profile is illustrated, but in the detection result display section 513, a plurality of response profiles indicating the detection results for each of the cells 42 used for the disease determination may be displayed. In the detection result display section 513, an image showing the state of the luminescence of the cell 42 in the olfactory sensor 4, which is imaged by the imaging device, may be displayed.

In a case where the response profiles relevant to the plurality of cells 42 are displayed in the detection result display section 513, the determination device 1 may set a display order on the screen, on the basis of the availability in the disease determination or the degree of similarity between each of the response profiles and the reference profile. The determination device 1, for example, preferentially displays the detection result of the cell 42 with high availability or a high degree of similarity in the detection result display section 513. The determination device 1 may display the detection results according to a predetermined number of cells 42 in descending order of the degree of priority, in the detection result display section 513. The determination device 1 may set the display order such that a detection result with a higher contribution ratio of the degree of similarity according to the cell 42 is preferentially displayed on the basis of the contribution ratio of the input information in the determination model described above. The contribution ratio, for example, can be calculated on the basis of a shapley additive explanation (SHAP) value, a Gini coefficient, local interpretable model-agnostic explanations (LIME), permutation feature importance (PFI), or the like.

Note that the detection result is not limited to being presented to the examinee via the terminal device 2. The determination device 1, for example, may output the detection result to another computer, a predetermined print device, or the like.

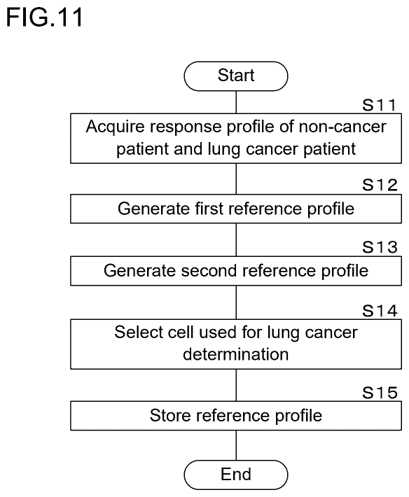

FIG. 11 is a flowchart illustrating an example of a processing procedure of generating the reference profile. The processing in the following flowchart is executed by the control unit 11, in accordance with the program 1P stored in the storage unit 12 of the determination device 1.

The control unit 11 of the determination device 1 acquires the response profile detected from the normal urine of the plurality of non-cancer patients and the response profile detected from the lung cancer urine of the plurality of lung cancer patients via the detection device 3 (step S11). The response profile, for example, is the time-dependent data of the luminescence intensity, and is generated for each of the cells 42 in the olfactory sensor 4. Information indicating the corresponding cell 42 may be associated with each of the response profiles.

The control unit 11 generates the first reference profile, on the basis of each of the response profiles with respect to the plurality of types of normal urine (step S12). The control unit 11, for example, calculates the geometric mean of the luminescence intensity detected from each normal urine for each elapsed time to generate the first reference profile.

The control unit 11 generates the second reference profile, on the basis of each of the response profiles with respect to the plurality of types of lung cancer urine (step S13). The control unit 11, for example, calculates the geometric mean of the luminescence intensity detected from each lung cancer urine for each elapsed time to generate the second reference profile.

The control unit 11 selects one or a plurality of cells 42 used for the lung cancer determination from the plurality of types of cells 42 included in the olfactory sensor 4 (step S14). The control unit 11, for example, calculates the maximum value or the intensity difference integral value of the luminescence intensity of each of the first reference profile and the second reference profile, for each of the cells 42 included in the olfactory sensor 4. The control unit 11 selects a cell 42 in which a difference in the calculated maximum value or the intensity difference integral value of the luminescence intensity is greater than or equal to the first threshold value set in advance, as the cell 42 for lung cancer determination. In step S14, the control unit 11 may specify the type and the number of cells 42 selected for the disease determination such that each determination accuracy is optimized on the basis of the determination accuracy of the possibility of the disease in the case of using each of the cells 42, and may select the cell 42 for lung cancer determination, in accordance with the specifying result.

The control unit 11 stores disease information, the cell information of the selected cell 42 for lung cancer determination, and the first reference profile and the second reference profile corresponding to the cell 42 in association with each other, in the detection DB 121 (step S15), and ends a series of processing.

The control unit 11 executes the processing described above on all diseases that can be the determination target, and generates and stores the first reference profile and the second reference profile for various types of disease determination.

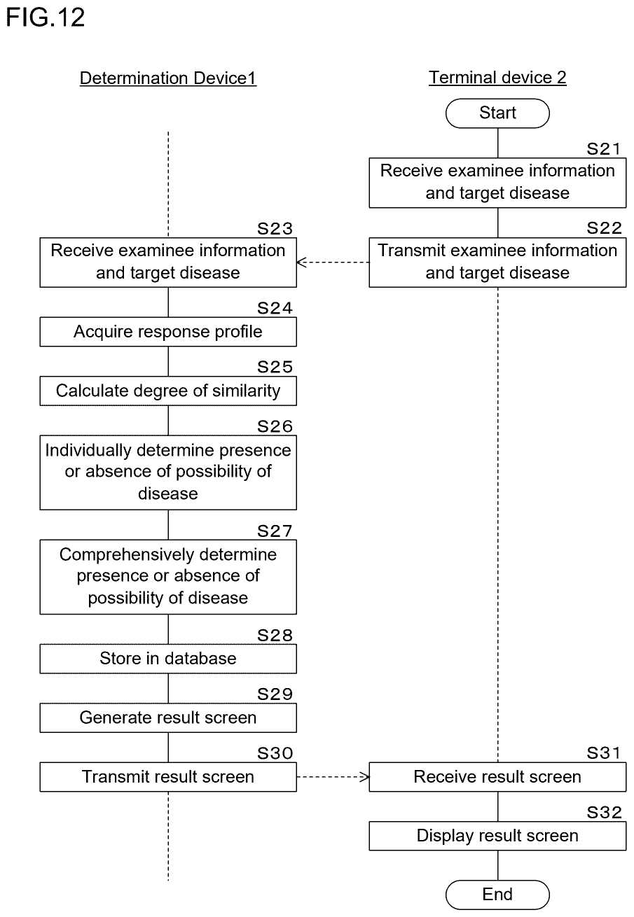

FIG. 12 is a flowchart illustrating an example of a processing procedure of determining the possibility of the disease. The following processing is executed by the control unit 11, in accordance with the program 1P stored in the storage unit 12 of the determination device 1, and is executed by the control unit 21, in accordance with the program 2P stored in the storage unit 22 of the terminal device 2.

The control unit 21 of the terminal device 2 receives the examinee information of the examinee who desires to be determined and the target disease, on the basis of the manipulation of the examinee using the reception screen (step S21). The control unit 21 transmits the received examinee information and target disease to the determination device 1 (step S22).

The control unit 11 of the determination device 1 receives the examinee information and the target disease (step S23).

The control unit 11 acquires the response profile generated from the urine sample derived from the examinee via the detection device 3 (step S24). The response profile is generated for each of the cells 42 in the olfactory sensor 4. The subject ID, the examinee ID, the sampling date, the detection device ID, the detection date, and the like are associated with the response profile. The control unit 11 may acquire the raw detection data obtained by detection from the detection device 3, and may perform various types of preprocessing on the acquired raw detection data to generate the response profile.

The control unit 11 calculates the degree of similarity between the response profile of the urine sample derived from the examinee and each of the first reference profile and the second reference profile, for each of the cells 42 for disease determination corresponding to the target disease (step S25).

The control unit 11 compares the calculated degrees of similarity, and specifies a health condition corresponding to a reference profile with a high degree of similarity as the health condition of the examinee to individually determine the presence or absence of the possibility of the disease in the examinee (step S26). In step S26, the presence or absence of the possibility of the disease is determined for each of the cells 42. The control unit 11, for example, comprehensively determines the presence or absence of the possibility of the disease by the majority rule of the individual determination for each of the cells 42 (step S27). The control unit 11 stores the subject ID, the examinee ID, the sampling date, the detection device ID, the detection date, the response profile for each type of cell, and the determination result in association with each other, in the detection DB 121 (step S28).

The control unit 11 generates the result screen showing the obtained determination result of the possibility of the disease (step S29). The control unit 11 transmits the generated result screen to the terminal device 2 corresponding to the examinee identified by the examinee information acquired in step S23 (step S30).

The control unit 21 of the terminal device 2 receives the result screen from the determination device 1 (step S31). The control unit 21 displays the received result screen on the display unit 24 (step S32), and ends a series of processing.

In the processing described above, it may be configured such that the user is capable of checking the determination result at an arbitrary timing by requesting the display of the result screen using the terminal device 2, and receiving the result screen in accordance with the request.

In the processing described above, the control unit 11 of the determination device 1 may update the reference profile, on the basis of the response profile and the determination result newly stored in the detection DB 121 by the processing of step 28. The control unit 11, for example, extracts one or a plurality of newly added response profiles from the detection DB 121, at suitable intervals. The control unit 11 regenerates the first reference profile, on the basis of a plurality of response profiles to which a response profile determined that there is no possibility of a cancer is newly added, among the extracted response profiles. Alternatively, the control unit 11 regenerates the second reference profile, on the basis of a plurality of response profiles to which a response profile determined that there is the possibility of a cancer is newly added, among the extracted response profiles.

In the above, the possibility of the disease is determined on the basis of the detection data with respect to the urine sample by using the olfactory sensor 4. The subject to be the analysis target is not limited to urine, and for example, may be blood, sweat, saliva, tears, exhalation, human skin gas, tissue fluid, joint fluid, follicular fluid, cerebrospinal fluid, seminal fluid, breast milk, vaginal fluid, and the like. In addition, the health condition to be the determination target is not limited to the possibility of the disease, and may be the possibility of other abnormalities, or the like. The determination of the health condition may be stress check, breath check, body odor check, or the like. The determination target of the health condition is not limited to humans, and may be animals.

According to this embodiment, the health condition can be determined on the basis of the detection data of the olfactory sensor 4, and the practicality of the olfactory sensor 4 is improved. By using the response profile indicating the response of the olfactory receptor, it is possible to accurately determine the health condition. The health condition is determined by comparison with the reference profile generated in advance, and the determination processing of the health condition is facilitated.

Since the examinee is capable of obtaining the determination result by submitting the subject or registering required information, a burden required for the examination is reduced, and the utilization rate of the service increases. Since it is possible to check the determination result by using the terminal device 2, it is possible to reliably grasp the determination result at an arbitrary timing. By displaying the detection result to be visually recognizable, in addition to the determination result, it is possible to more reliably and detailedly check the result. By displaying the reference profile to be the determination reference, in addition to the own detection result of the examinee, explainability with respect to the determination result is improved.

### Second Embodiment

In a second embodiment, correction for eliminating an individual difference of the response profile is performed. In each of the following embodiments, differences from the first embodiment will be mainly described, and the same reference numerals will be applied to the same configurations as those in the first embodiment and the detailed description thereof will be omitted.

The response signal detected from the subject may have an individual difference due to various factors. For example, even in the case of urine samples containing odor compounds with the same concentration, the value of luminescence intensity detected from a urine sample containing impurities is greater or less than that of a urine sample not containing impurities due to the influence of impurities contained in the urine sample. That is, an individual difference occurs in the correlation between the concentration of the odorous molecules and the luminescence intensity due to the influence of the impurities. In addition, the response signal may cause an individual difference due to the influence of the balance of the odorous molecules. The individual difference occurs not only in the luminescence intensity but also in various response signals.

In the case of executing the determination based on the response profile, the occurrence of the individual difference as described above leads to a decrease in the determination accuracy. In particular, as described in the first embodiment, in the case of determining whether the urine sample is similar to the profile of the normal urine or the profile of the lung cancer urine by comparing the response profile to be the target with the reference profile, the possibility of erroneous determination due to the influence of the individual difference increases. In this embodiment, by performing correction processing for eliminating the individual difference in the response profile, the determination accuracy is improved.

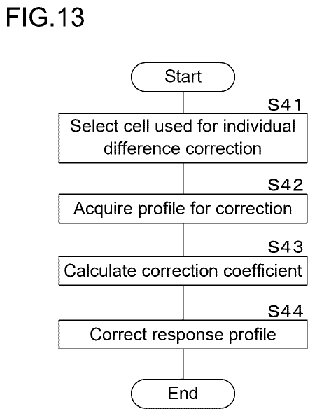

FIG. 13 is a flowchart illustrating an example of a processing procedure executed by the determination device 1 of the second embodiment. The processing of FIG. 13, for example, is executed between step S24 and step S25 of the first embodiment.

The control unit 11 of the determination device 1 selects one or a plurality of cells 42 used for the correction of the individual difference, from the plurality of types of cells 42 included in the olfactory sensor 4 (step S41). As the cell for individual difference correction, the cell 42 of which the reactivity according to the target disease (for example, a lung cancer) is lower than that of the cell for disease determination is preferable. As the cell for individual difference correction, a cell having reactivity that is not significantly changed in accordance with the presence or absence of the lung cancer and not having a significant difference in the response profile is more preferable. The cell for individual difference correction may be a cell that reacts with odorous molecules contained in both of the normal urine and the lung cancer urine at approximately the same concentration, may be a cell that reacts with odorous molecules artificially added to urine, which are not contained in the urine before being added.

The control unit 11, for example, the cell 42 for individual difference correction is selected on the basis of the degree of dissimilarity between the first reference profile based on non-cancer urine and the second reference profile based on lung cancer urine. Specifically, the control unit 11 calculates the degree of dissimilarity between the first reference profile and the second reference profile, for each of the cells 42 included in the olfactory sensor 4. The control unit 11 selects a cell 42 of which the calculated degree of dissimilarity is less than a second threshold value set in advance, as the cell 42 for individual difference correction. The degree of dissimilarity between the first reference profile and the second reference profile, for example, may be a difference between the maximum value of the luminescence intensity in the first reference profile and the maximum value of the luminescence intensity in the second reference profile, or an intensity difference integral value between the first reference profile and the second reference profile, as with the case of specifying the cell 42 for disease determination. Note that in the above, the degree of dissimilarity between the first reference profile and the second reference profile is used, but the degree of similarity may be used.

It is preferable that each of the first reference profile and the second reference profile is generated on the basis of response profiles of a plurality types of urine obtained from a plurality of people. The second threshold value may be a value that is the same as the first threshold value used for the selection of the cell 42 for disease determination or less than the first threshold value. The cell 42 for individual difference correction may be selected from the cells 42 other than the cell 42 for disease determination, among the cells 42 in the olfactory sensor 4. Note that in a case where the type of cell 42 for individual difference correction is well-known, the selecting step described above may be omitted.

The control unit 11 acquires a profile for correction corresponding to the selected cell 42 for correction (step S42). The profile for correction may be a response profile generated on the basis of the statistical value of the first reference profile and the second reference profile. In a case where the maximum value or the intensity difference integral value of the luminescence intensity of the first reference profile and the second reference profile is approximately zero, either the first reference profile or the second reference profile may be used as the profile for correction. The profile for correction is acquired for each of the cells 42 for correction.

The control unit 11 calculates a correction coefficient (a correction value) for correcting the response profile, on the basis of the acquired profile for correction, and the response profile of the urine sample of the examinee by the cells 42 for correction (step S43).

The correction coefficient, for example, can be calculated by the following method. An area surrounded by each of the response profile of the urine sample of the examinee by the cells 42 for correction and the profile for correction, and an x axis (a time axis) is divided at predetermined time intervals. A ratio between the area of a first region by the response profile and the area of a second region surrounded by the profile for correction is obtained for each division. The geometric mean value of the ratios in all of the divisions is set as the correction coefficient. Note that a method for calculating the correction coefficient is not limited to the example described above, and may be set such that the individual difference of the response profile can be corrected. The correction coefficient may be adjusted in accordance with the type of cell 42 for determination. For example, by multiplying the correction coefficient calculated on the basis of the profile for correction and the response profile and a predetermined coefficient set in advance for each of the cells 42 for determination, the correction coefficient for each of the cells 42 for determination may be obtained.

In a case where a plurality of cells 42 are selected as the cell 42 for correction, the control unit 11 may calculate the correction coefficient described above for each of the cells 42 for correction, and may obtain the statistical value (for example, the geometric mean, the median value, or the like) of each of the calculated correction coefficients to set the final correction coefficient.

The control unit 11 corrects the response profile of each of the cells 42 selected for the disease determination by using the calculated correction coefficient (step S44). Specifically, by multiplying each luminescence intensity of the response profile for disease determination and the correction coefficient, the response profile after correction is generated. The control unit 11 executes processing subsequent to step S25 by using the response profile after correction to determine the possibility of the disease, on the basis of the response profile after correction. The control unit 11 may store the calculated correction coefficient and the response profile after correction in the detection DB 121.

According to the processing described above, it is possible to obtain the correction coefficient on the basis of the response profile of the cell 42 for correction selected from the cells 42 in the olfactory sensor 4 and the reference profile, and corrects the response profile of the cell 42 for disease determination by using the obtained correction coefficient. In the cell 42 for correction, different characteristics of the response signal according to a specific characteristic (for example, the presence or absence of the possibility of a disease) are not exhibited, and the response profile hardly depends on the specific characteristic. On the other hand, in the cell 42 for disease determination, different characteristics of the response signal according to the specific characteristic are exhibited, and the response profile strongly depends on the specific characteristic.

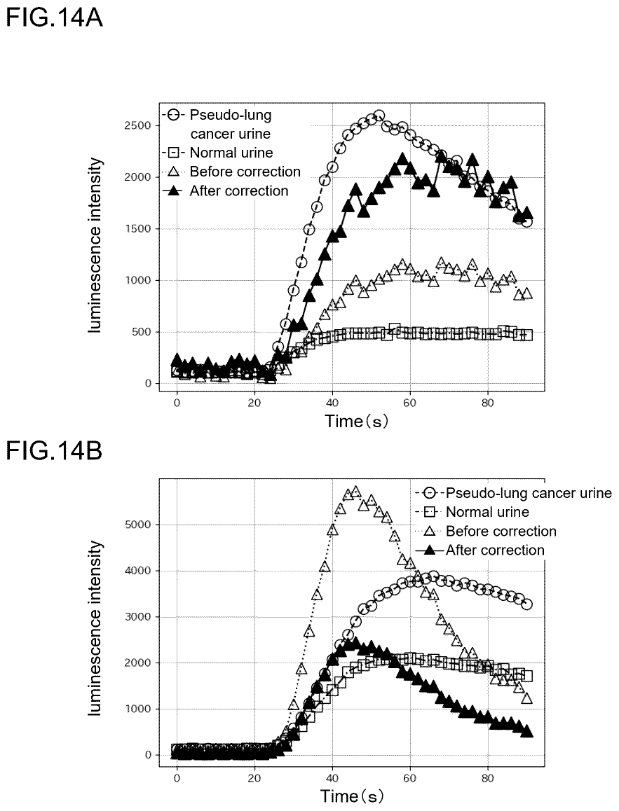

FIG. 14 is a diagram illustrating an example of the response profile according to the presence or absence of the correction processing. In the example illustrated in FIG. 14A, the luminescence intensity of the response profile before the correction is generally decreased by the influence of impurities that weaken the activity. The response profile after correction is corrected such that the luminescence intensity increases. By the correction illustrated in FIG. 14A, it is possible to suppress a possibility that a subject with the possibility of a disease is erroneously determined as not having the possibility of the disease.

In the example illustrated in FIG. 14B, the luminescence intensity of the response profile before correction is generally increased by the influence of impurities that strengthen the activity. The response profile after correction is corrected such that the luminescence intensity decreases. By the correction illustrated in FIG. 14B, it is possible to suppress a possibility that a subject without the possibility of a disease is erroneously determined as having the possibility of the disease.

According to this embodiment, it is possible to correct the individual difference of the subject, and suppress a decrease in the determination accuracy due to the individual difference. In a case where a biological sample is measured by utilizing a biosensor using a biological element, it is considered that a difference may occur in the response signal due to various factors. According to this embodiment, it is possible to suitably eliminate such an individual difference.

According to this embodiment, it is possible to efficiently set the cell for individual difference correction, on the basis of the feature of the response profile. By preparing the biosensor in which a plurality of sensor cells are arranged, and acquiring the response profile of each cell, it is possible to select a suitable cell for individual difference correction, in accordance with various characteristics to be determined.

### Third Embodiment

In a third embodiment, the possibility of a disease is determined by using a learning model.

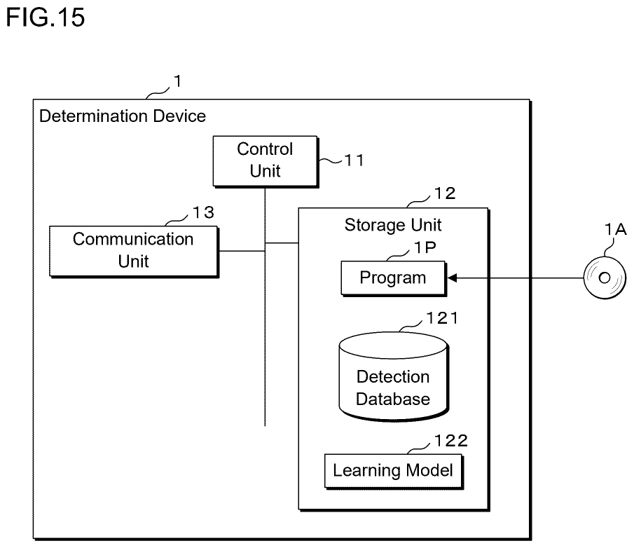

FIG. 15 is a block diagram illustrating a configuration example of the determination device 1 of the third embodiment. The determination device 1 of the third embodiment stores a learning model 122 in the storage unit 12. The learning model 122 is a machine learning model that has learned predetermined training data. It is assumed that the learning model 122 is used as a program module configuring a part of artificial intelligence software.

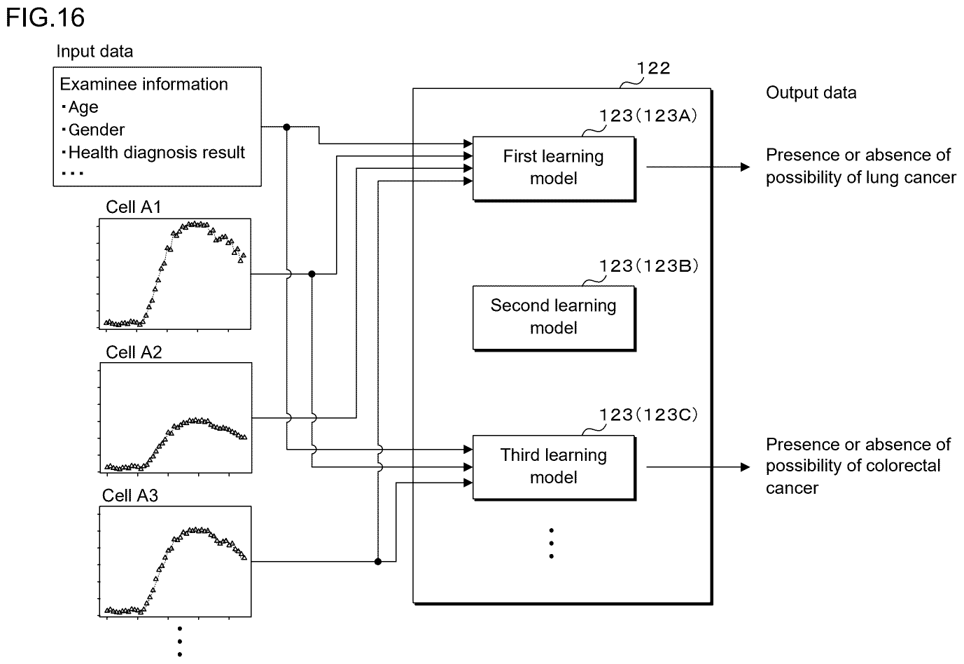

FIG. 16 is an explanatory diagram illustrating the outline of the learning model 122. The learning model 122 receives the response profile of the luminescence intensity detected from the urine sample of the examinee as input, and outputs information indicating the presence or absence of the possibility of the target disease with respect to the response profile. The learning model 122, for example, is a convolutional neural network (CNN) that is one type of neural network.

The learning model 122 includes an input layer to which the response profile is input, an output layer that outputs the presence or absence of the possibility of the disease, and an intermediate layer (a hidden layer). The intermediate layer may include a convolution layer, a pooling layer, a fully connected layer, and the like. The intermediate layer has a plurality of nodes extracting the feature amount of the response profile, and transfers the extracted feature amount to the output layer by using various parameters. In a case where the response profile is input to the input layer, arithmetic is performed in the intermediate layer by learned parameters, and output information indicating a classification result of the presence or absence of the possibility of the target disease is output from the output layer.

The learning model 122 can be generated by preparing the training data associated with a label indicating the presence or absence of the possibility of the target disease with respect to the response profile, and performing machine-learning on the unlearned neural network with training data. As a ground truth label, for example, a diagnosis result of an experienced medical doctor is used. The training data includes response profiles detected from the subjects of a plurality of examinees with the possibility of a disease, and response profiles detected from the subjects of a plurality of examinees without the possibility of the disease. The learning model 122 is trained with the relationship between the response profile and the possibility of the disease.

The determination device 1 inputs a plurality of response profiles included in the training data to an input layer of an unlearned neural network model, and acquires the presence or absence of the possibility of the disease output from the output layer via the arithmetic processing in the intermediate layer. The determination device 1 compares the presence or absence of the possibility of the disease output from output layer with the presence or absence of the possibility of the disease included in the training data, and optimizes parameters such as the weight between neurons, for example, by using backpropagation, such that the presence or absence of the possibility of the disease output from the output layer approaches to a ground truth value. Note that the learning model 122 may be constructed by an external device, and may be deployed in the determination device 1.

The input data that is input to the learning model 122 is not limited to the image representing the response profile of the luminescence intensity, and may be the value of the time-dependent luminescence intensity. Further, the input data of the learning model 122 may include the type of cell 42 corresponding to the luminescence intensity. It is obvious that the input to the learning model 122 may be a response signal other than the luminescence intensity.

The output layer of the learning model 122 is not limited to performing estimation in accordance with the presence or absence of the possibility of the disease, and for example, may output a possibility level that is classified into a plurality of levels in accordance with the degree of possibility, or may output a numerical value indicating the possibility in a percentage.

In the learning model 122, one model may be constructed for each type of cell 42, one model may be constructed for each type of disease, or one model may be constructed for each combination between the cell 42 and the disease. The learning model 122 may receive response signals relevant to a plurality of types of cells 42 as input, and may estimate the possibility of a disease corresponding to the response signals relevant to the plurality of types of cells 42.

The learning model 122 may receive response profiles with respect to subjects sampled from the examinee on a plurality of sampling dates as input, and may output the possibility of a disease. In this case, the learning model 122 receives the latest response profile and the past several response profiles as input, and may output current possibility of the disease in the examinee, or may output the future possibility of the disease.

The configuration of the learning model 122 is not limited to the example described above insofar as the possibility of the target disease can be identified with respect to the time-dependent data of the response signal. The learning model 122, for example, may be a model constructed by other learning algorithms, such as a recurrent neural network (RNN), a graph neural network (GNN), Transformer, a support vector machine (SVM), logistic regression, and extreme gradient boosting (XGBoost).

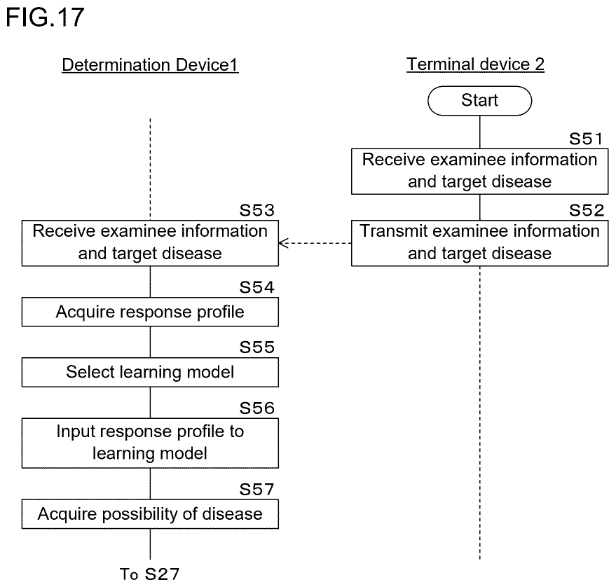

FIG. 17 is a flowchart illustrating an example of a processing procedure executed by the determination system 100 of the third embodiment.

The control unit 21 of the terminal device 2 executes the same processing as that of step S21 to step S22, and receives the examinee information and the target disease (step S51) and transmits the examinee information and the target disease (step S52).

The control unit 11 of the determination device 1 executes the same processing as that of step S23 to step S24, receives the examinee information and the target disease (step S53), and acquires the response profile (step S54).

The control unit 11 selects the learning model 122 according to the cell 42 for disease determination corresponding to the received target disease and target disease, from a plurality of learning models 122 stored in the storage unit 12 (step S55).

The control unit 11 inputs the response profile of the corresponding cell 42 for disease determination to each of the selected learning models 122 (step S56). The control unit 11 acquires the possibility of the disease output from the learning model 122 (step S57). The learning model 122, for example, outputs the presence or absence of the possibility for each type of cell 42 and disease. Thereafter, the control unit 11 executes the same processing as that of step S27 to step S31.

In the processing described above, as with the first embodiment, the control unit 11 may comprehensively determine the possibility of the disease by using the determination model. In this case, the determination model may be configured to receive the possibility of the disease for each type of cell 42 output from the learning model 122 as input, and output the possibility of the disease.

According to this embodiment, it is possible to easily and accurately determine the possibility of the disease by using the learning model 122.

Regarding the embodiments described above, the following appendices will be further disclosed.

### Appendix 1

A determination method causing a computer to execute processing of:
acquiring a time-dependent response signal with respect to a target subject derived from a determination target, detected by using an olfactory sensor including cells that have olfactory receptors and respond to odorous molecules; and
determining a health condition of the target, on the basis of a profile of the acquired time-dependent response signal with respect to the target subject, and a with a first profile obtained from a time-dependent response signal with respect to a subject derived from a target with a favorable health condition and a second profile obtained from a time-dependent response signal with respect to a subject derived from a target with an unfavorable health condition.

### Appendix 2

The determination method according to Appendix 1,
in which the first profile is generated on the basis of a mean value or a median value of each response signal of a plurality of subjects derived from the target with a favorable health condition, and the second profile is generated on the basis of a mean value or a median value of each response signal of a plurality of subjects derived from the target with an unfavorable health condition.

### Appendix 3

The determination method according to Appendix 1 or Appendix 2,
in which a degree of similarity between the profile of the response signal of the target subject, and each of the first profile and the second profile is acquired, and
a health condition corresponding to a profile with a high degree of similarity that is acquired is determined as the health condition of the determination target.

### Appendix 4

The determination method according to any one of Appendix 1 to Appendix 3,
in which the olfactory sensor includes a plurality of cells having different olfactory receptors, and
the health condition of the target is comprehensively determined by integrating the health conditions individually determined for each profile of the response signal relevant to each cell.

### Appendix 5

The determination method according to any one of Appendix 1 to Appendix 4,
in which the health condition of the target is acquired on the basis of a learning model outputting a health condition of the subject when the profile of the response signal of the subject is input.

### Appendix 6

The determination method according to any one of Appendix 1 to Appendix 5,
in which the profile of the response signal of the target subject is corrected by using a predetermined correction method, and
the health condition of the target is determined on the basis of the corrected profile of the response signal of the target subject, and the first profile and the second profile.

### Appendix 7

The determination method according to any one of Appendix 1 to Appendix 6,
in which the olfactory sensor includes a plurality of cells having different olfactory receptors,
the first profile and the second profile relevant to each cell are acquired, and
a specific cell used for determination of the health condition is selected from the plurality of cells in the olfactory sensor, on the basis of the acquired first profile and second profile for each cell.

### Appendix 8

The determination method according to any one of Appendix 1 to Appendix 7,
in which screen information displaying a determination result of the health condition of the target, the profile of the response signal of the target subject, and the first profile and the second profile is generated.

### Appendix 9

The determination method according to any one of Appendix 1 to Appendix 8,
in which the olfactory receptor is an insect olfactory receptor, and determines the presence or absence of a disease in the target.

The embodiments disclosed herein should be considered as illustrative in all respects and not restrictive. The technical features described in each example can be combined with each other, and the scope of the invention is intended to include all modifications within the claims and the scope equivalent to the claims.

The sequence described in each of the embodiments is not limited, and each processing procedure may be executed by changing the order thereof, and a plurality of types of processing may be executed in parallel, within the bounds of consistency. The main agent performing each type of processing is not limited, and the processing of each device may be executed by another device, within the bounds of consistency.

The respects described in each of the embodiments can be combined with each other. In addition, the independent claims and the dependent claims set forth in the claims can be combined with each other in any and all combinations, regardless of the format of reference. Further, the claims are in a format in which a claim refers to two or more other claims (the format of a multiple dependent claim), but are not limited thereto. The claims may be in a format in which a multiple dependent claim refers to at least one of multiple dependent claims (a multiple-multiple dependent claim).

It is to be noted that, as used herein and in the appended claims, the singular forms “a”, “an”, and “the” include plural referents unless the context clearly dictates otherwise.

What is claimed is:

1. A determination method executed on a computer, comprising:
acquiring a time-dependent response signal with respect to a target subject derived from a determination target, detected by using an olfactory sensor including cells that have olfactory receptors and respond to odorous molecules; and
determining a health condition of the determination target, on the basis of a profile of the acquired time-dependent response signal with respect to the target subject, and a first profile obtained from a time-dependent response signal with respect to a subject derived from a target with a favorable health condition and a second profile obtained from a time-dependent response signal with respect to a subject derived from a target with an unfavorable health condition.

2. The determination method according to claim 1,
wherein the first profile is generated on the basis of a mean value or a median value of each response signal of a plurality of subjects derived from the target with a favorable health condition, and the second profile is generated on the basis of a mean value or a median value of each response signal of a plurality of subjects derived from the target with an unfavorable health condition.

3. The determination method according to claim 1, further comprising:
acquiring a degree of similarity between the profile of the response signal of the target subject and each of the first profile and the second profile; and
determining a health condition corresponding to a profile with a high degree of similarity that is acquired, as the health condition of the determination target.

4. The determination method according to claim 1,
wherein the olfactory sensor includes a plurality of cells having different olfactory receptors, and
the method further comprising
comprehensively determining the health condition of the determination target by integrating the health conditions individually determined for each profile of the response signals relevant to each cell.

5. The determination method according to claim 1, further comprising
acquiring the health condition of the determination target, on the basis of a learning model outputting a health condition of the subject when the profile of the response signal of the subject is input.

6. The determination method according to claim 1, further comprising:
correcting the profile of the response signal of the target subject by using a predetermined correction method; and
determining the health condition of the determination target, on the basis of the corrected profile of the response signal of the target subject, and the first profile and the second profile.

7. The determination method according to claim 1,
wherein the olfactory sensor includes a plurality of cells having different olfactory receptors, and
the method further comprising:
acquiring the first profile and the second profile relevant to each cell; and
selecting a specific cell used for determination of the health condition from the plurality of cells in the olfactory sensor, on the basis of the acquired first profile and second profile for each cell.

8. The determination method according to claim 1, further comprising
generating screen information displaying a determination result of the health condition of the determination target, the profile of the response signal of the target subject, and the first profile and the second profile.

9. The determination method according to claim 1,
wherein the olfactory receptor is an insect olfactory receptor, and determines the presence or absence of a disease in the determination target.

10. A determination device, comprising
a processor configured to:
acquire a time-dependent response signal with respect to a target subject derived from a determination target, detected by using an olfactory sensor including cells that have olfactory receptors and respond to odorous molecules; and
determine a health condition of the determination target, on the basis of a profile of the acquired time-dependent response signal with respect to the target subject, and a first profile obtained from a time-dependent response signal with respect to a subject derived from a target with a favorable health condition and a second profile obtained from a time-dependent response signal with respect to a subject derived from a target with an unfavorable health condition.

11. A non-transitory computer-readable recording medium recording a computer program causing a computer to execute processing of:
acquiring a time-dependent response signal with respect to a target subject derived from a determination target, detected by using an olfactory sensor including cells that have olfactory receptors and respond to odorous molecules; and
determining a health condition of the determination target, on the basis of a profile of the acquired time-dependent response signal with respect to the target subject, and a first profile obtained from a time-dependent response signal with respect to a subject derived from a target with a favorable health condition and a second profile obtained from a time-dependent response signal with respect to a subject derived from a target with an unfavorable health condition.
